(19) United States
(12) Patent Application Publication
(10) Pub. No.: US 2012/0015026 A1
(43) Pub. Date: Jan. 19, 2012

(54) PHARMACEUTICAL COMPOSITION CONTAINING A DRUG AND SIRNA

(76) Inventors: Giancarlo Francese, Basel (CH); Michael Keller, Basel (CH)

(21) Appl. No.: 13/258,919

(22) PCT Filed: Mar. 24, 2010
(86) PCT No.: PCT/EP10/53802

§ 371 Date: Sep. 22, 2011

(30) Foreign Application Priority Data
Mar. 25, 2009 (EP) 09156232.2

(51) Int. Cl. A61K 9/127; A61K 33/24; A61K 38/12; A61P 35/00; A61K 38/14; A61K 38/13; A61K 38/17; A61K 38/06; A61K 31/7088; A61K 31/704

(52) U.S. Cl. 424/450; 514/44 A; 424/649; 514/21.1; 514/34; 514/20.9; 514/20.5; 514/15.3; 514/21.9

(57) ABSTRACT

The present invention relates generally to the fields of molecular biology, medicine, oncology, and delivery of therapeutic compounds. In particular, the present invention relates to pharmaceutical compositions containing a hydrophobic drug substance and an inhibitory nucleic acid molecule, such as short interfering RNA (siRNA), in a single drug delivery system, as well as a process for making and a process for administering the same.

### FIELD OF INVENTION

The present invention relates generally to the fields of molecular biology, medicine, oncology, and delivery of therapeutic compounds. In particular, the present invention relates to pharmaceutical compositions containing a hydrophobic drug substance and an inhibitory nucleic acid molecule, such as short interfering RNA (siRNA), in a single drug delivery system, as well as a process for making and a process for administering the same.

### BACKGROUND OF THE INVENTION

There is an ever increasing number of drug substances which are poorly soluble in water but highly soluble in organic solvents EPO906 (epothilone B) is a patupilone that falls into such a category; it is poorly soluble in water (<0.1 mg/l) but highly soluble in organic solvents such as ethanol, ethylacetate, dichloromethane, etc. Poorly water soluble, i.e., hydrophobic, drug substances provide challenges to delivery in an injectable form such as through parenteral administration. Formulations which include these drugs must be capable of presenting a therapeutically effective amount of the drug to the desired absorption site in an absorbable form, Pharmaceutical compositions for delivery of poorly soluble drugs must carry the drug through the aqueous environment, while maintaining the drug in an absorbable form, and while avoiding the use of physiologically harmful solvents or excipients. A number of approaches to formulating hydrophobic drugs for oral or parenteral delivery are known such as formulations in which the drug is present in an oil-in-water emulsion, a microemulsion, or a solution of micelles, liposomes, or other multi-lamellar carrier particles. Liposomes are microscopic vesicles made up of amphipathic molecules such as lipids, containing both hydrophobic and hydrophilic regions and can enclose an aqueous volume.

Short interfering RNAs (siRNAs) are double stranded ribonucleotide sequences of 19-27 base pairs in length, highly negatively charged, and soluble predominantly in water. siRNAs are responsible for RNA interference, the process of sequence-specific post-transcriptional gene silencing in animals and plants. siRNAs are generated by ribonuclease III cleavage from longer double-stranded RNA (dsRNA) which are homologous to the silenced gene or by delivering synthetic RNAs to cells. Delivery of siRNA in viva is known to be very difficult, thus, limiting the therapeutic potential of siRNA. For example, delivery methods that are effective for other types of nucleic acid molecules are not necessarily effective for siRNA Most studies using siRNA in viva involve manipulation of gene expression in a cell line prior to introduction into an animal model or incorporation of siRNA into a viral vector. Delivery of “naked” siRNA in vivo has been restricted to site-specific injections or through high-pressure means that are not clinically practical. Further, systemically administered siRNA is rapidly cleared by the kidneys or liver due to its high solubility in water and negative charge. Therefore, it is preferable to encapsulate siRNAs into a drug delivery system (DDS) that enhances its circulation time in the body and prevents degradation by extracellular nucleases.

Prior combination approaches for treating cancer involve a separate administration of two synergistically acting drugs using two separate formulation modes to deliver the two active drug substances. For instance, WO 2006113679 discloses siRNA formulated within neutral liposomes for a first administration and a second, separate administration of a chemotherapeutic agent (e.g. doxorubicin etc.) which may take place prior to, simultaneously with, or after the first administration. The existing prior art describes the use of an individually tailored DOS for each drug substance resulting in inconvenient, multiple steps of administering the drug substances,

There is a need therefore, for a convenient, single drug delivery system comprising a siRNA and hydrophobic drug substance The present invention fulfills this need by providing a vesicular drug delivery system (VDDS) comprising at least two drug substances: a nucleic acid molecule such as siRNA and a hydrophobic drug. The present invention further provides a process of making the VDDS and a process for administering the VDDS.

### BRIEF DESCRIPTION OF THE DRAWINGS

FIG, 1 illustrates a synthetic reaction scheme for synthesis of liposomes comprising a nucleic acid siRNA and a hydrophobic drug substance, EPO906.

FIG. 2 illustrates an arrangement of the vesicular drug delivery system showing a hydrophobic drug EPO906 incorporated in the lipid bilayer and a nucleic acid siRNA encapsulated by the liposome in the aqueous enclosure with an optional targeting moiety, folate-PEG-lipid attached to the drug delivery system.

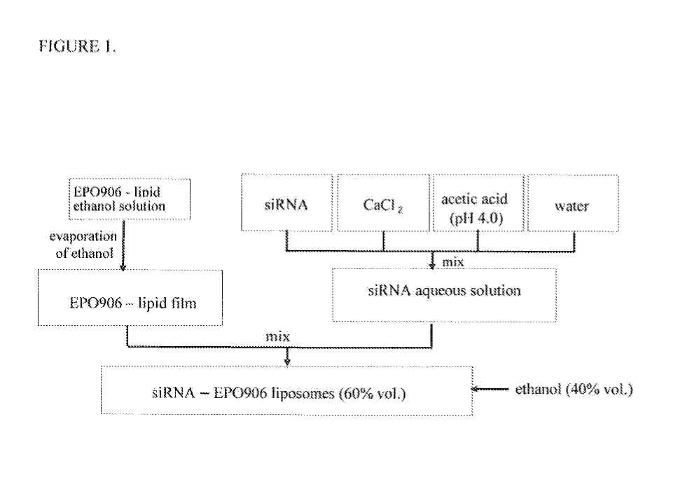

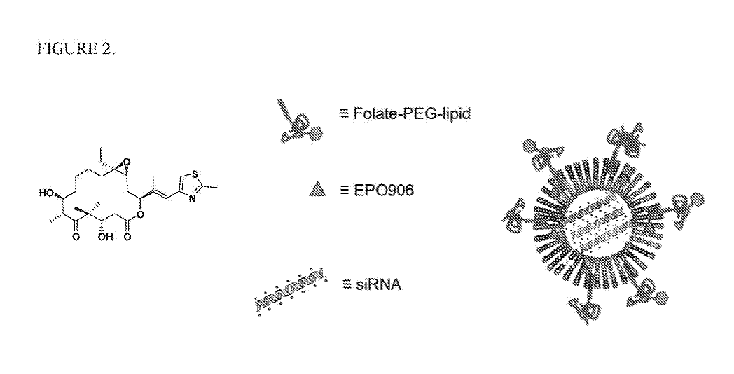

### SUMMARY OF THE INVENTION

The present invention is a vesicular drug delivery system (VDDS) comprising: (a) at least one lipid bilayer enclosing at least one aqueous cavity; (b) a short interfering ribonucleic acid (siRNA) molecule contained within said at least one aqueous cavity; and (c) at least one hydrophobic drug substance embedded in said at least one lipid bilayer. In accordance with the present invention; the lipid bilayer comprises a neutral lipid that is phosphatidylcholine. The present invention further includes an embodiment in which the lipid bilayer comprises a combination of phosphatidylcholine and at least one additional neutral lipid, cationic lipid and/or anionic lipid.

The present invention further includes an embodiment in which a “targeting moiety” or ligand attached to the VDDS for recognizing target tissue to improve intracellular delivery of drug substances delivered by VDDSs. The targeting moiety may be coupled to a hydrophilic polymer (e.g., PEG).

The present invention provides a method of making a VDDS comprising (a) at least one lipid bilayer enclosing at least one aqueous cavity, wherein the lipid comprises phosphatidylcholine; (b) at least one short interfering ribonucleic acid (siRNA) contained within said at least one aqueous cavity; and (c) at least one hydrophobic drug substance embedded in said at least one lipid Weyer. The method comprises the steps of (i) forming a first solution comprising a neutral lipid phosphatidylcholine, at least one hydrophobic drug and a water-miscible organic solvent, (ii) forming a lipid film by evaporating the water-miscible organic solvent from the first solution, (iii) forming a second solution comprising at least one short interfering ribonucleic acid (siRNA) and an aqueous solution, (iv) combining the lipid film of (ii) with the second solution of (iii) to form the VDDS.

The present invention further provides a method of making a VDDS comprising (a) at least one lipid bilayer enclosing at least one aqueous cavity, wherein the lipid comprises a combination of a neutral lipid phosphatidylcholine and at least one additional neutral lipid, cationic lipid and/or anionic lipid; (b) at least one short interfering ribonucleic acid (siRNA) contained within said at least one aqueous cavity; and (c) at least one hydrophobic drug substance embedded in said at least one lipid bilayer. The method comprises the steps of: (i) forming a first solution comprising the neutral lipid phosphatidylcholine, the additional neutral or cationic or anionic lipid(s), at least one hydrophobic drug and a water-miscible organic solvent, (ii) forming a lipid film by evaporating the water-miscible organic solvent from the first solution, (iii) forming a second solution comprising at least one short interfering ribonucleic acid (siRNA) and an aqueous solution, (iv) combining the lipid film of (ii) with the second solution of (iii) to form the VDDS.

The present invention further provides a method of simultaneous co-delivery of at least one hydrophobic drug substance and at least one siRNA to a subject, said method comprising administering to the subject a vesicular drug delivery system which comprises: (a) at least one lipid bilayer enclosing at least one aqueous cavity; (b) at least one short interfering ribonucleic acid (siRNA) contained within said at least one aqueous cavity; and (c) at least one hydrophobic drug substance embedded in said at least one lipid bilayer. In accordance with the present invention, the lipid bilayer or bilayers are formed by at least one neutral lipid that is phosphatidylcholine and, optionally, at least one additional neutral lipid, cationic lipid and/or anionic lipid.

### DETAILED DESCRIPTION OF THE INVENTION

The present invention relates to a pharmaceutical composition containing a hydrophobic drug substance and an inhibitory nucleic acid molecule, such as short interfering RNA (siRNA), in a single drug delivery system (DDS). The DOS of the present invention eliminates the need for separate administrations of a hydrophobic drug and inhibitory nucleic acid molecules, such as siRNA, resulting in enhanced patient compliance to a treatment regimen.

As used herein, the term “drug delivery system” or “DDS” means a pharmaceutical composition containing at least one therapeutic drug substance or nucleic acid molecule to be administered to a mammal, e.g., a human. A pharmaceutical composition is “pharmaceutically acceptable” which refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk.

As used herein, the term “drug substance” means any compound, substance, medicament, nucleic acid or amino acid sequence, or active ingredient having a therapeutic or pharmacological effect, and which is suitable for administration to a mammal. This term can refer to either or both short interfering ribonucleic acids (“siRNAs”) and hydrophobic drugs.

As used herein, the term “short interfering RNA” or “siRNA” means double stranded ribonucleotide sequences of typically 15-50 base pairs and preferably 19-27 base pairs in length that are highly negatively charged and soluble predominantly in water. siRNA may be composed of either two annealed ribonucleotide sequences or a single ribonucleotide sequence that forms a hairpin structure. One of ordinary skill would understand that siRNA is responsible for RNA interference, the process of sequence-specific post-transcriptional gene silencing in animals and plants, siRNAs are generated by ribonuclease Hi cleavage from longer double-stranded RNA (dsRNA) which are homologous to the silenced gene or by delivering synthetic RNAs to cells. Techniques for the design of such molecules for use in targeted inhibition of gene expression are well known to one of skill in the art.

The drug delivery system of the present invention is a vesicular drug delivery system (“VDDS”) comprising: (a) at least one lipid bilayer enclosing at least one aqueous cavity: (b) a short interfering ribonucleic add (siRNA) molecule contained within said at least one aqueous cavity; and (c) at least one hydrophobic drug substance embedded in said at least one lipid bilayer. The lipid bilayer or bilayers of the present invention are formed by at least one neutral lipid that is phosphatidylcholine and, optionally, at least one additional neutral lipid, cationic lipid and/or anionic lipid.

In one embodiment of the invention, the VOIDS campuses : (a) at least one lipid bilayer enclosing one aqueous cavity, wherein the lipid is comprised of a neutral lipid phosphatidylcholine and optionally, at least one additional neutral lipid, cationic lipid and/or anionic lipid; (b) a short interfering ribonucleic acid (siRNA) molecule contained within the one aqueous cavity, and (c) a hydrophobic drug substance embedded in the lipid bilayer.

In another embodiment of the invention, the VDDS is made up of a multilamellar vesicle (i.e., a liposome with multiple bilayers) comprising: (a) several lipid bilayers enclosing several aqueous cavities, wherein the lipid is comprised of a neutral lipid phosphatidylcholine or a combination of a neutral lipid phosphatidylcholine and at least one additional neutral lipid; (b) a short interfering ribonucleic acid (siRNA) molecule contained within each aqueous cavity; and (c) a hydrophobic drug substance embedded in the several lipid bilayers.

In any of the foregoing embodiments, the various components may be arranged according to convenience and need. For example, a siRNA having a particular nucleotide sequence may be encapsulated in an aqueous cavity of the VDDS. Alternatively, various siRNA molecules having different nucleotide sequences may be encapsulated in the aqueous cavity or cavities of the VDDS. Likewise, one particular hydrophobic drug may be embedded in the lipid bilayer, or various different hydrophobic drugs may be embedded in the lipid bilayer.

The VDDS of the present invention comprises one or multiple bilayers of a lipid, preferably comprising at least one neutral lipid that is phosphatidylcholine, that form a liposome. Examples include, unilamellar vesicular drug delivery system (ULVDDS) or a multilamellar vesicular drug delivery system (MLVDDS). A ULVDDS contains one single lipid bilayer surrounding an aqueous cavity while a MLVDD contains multiple aqueous compartments and bilayers. The drug substances of the present invention can be included in an MLVDDS in order to provide for time-release of the drug substances once the hydrophilic drug substances entrapped in the aqueous fluids between bilayers and/or the hydrophobic drug substances incorporated in the bilayers are released as each bilayer degrades. The VDDS of the present invention are expected to have a particle size ranging between 50 and 250 nm, preferably between 50 and 150 nm, and most preferably between 50 and 100 nm.

Liposome-forming lipids are well known and generally include phospholipids, with net neutral or negative charge. The term “phospholipid” refers to a hydrophobic molecule comprising at least one phosphorus group which can be natural or synthetic. For example, a phospholipid can comprise a phosphorus-containing group and saturated or unsaturated alkyl group, optionally substituted with OH, COOH, oxo, amine, or substituted or unsubstituted aryl groups. Phospholipids differ from each other in length and degree of saturation of their acylic chains. One of ordinary skill would understand that phospholipids can be selected based on the size of the final VDDS desired.

Cationic liposomes are described in PCI publications WO02/100435A1, WO03/015757A1, and WO0402921 3A2: U.S. Pat. Nos. 5,962,016, 5,030,453, and 6,680,068; and U.S. Patent App. No 2004/0208921. Furthermore, neutral lipids have been incorporated into cationic liposomes which have been used to deliver siRNA to various cell types.

The liposome-forming lipid or lipids used the present invention includes at least one neutral lipid that is phosphatidylcholine. The term “phosphatidylcholine” as used herein refers to both phosphatidylcholine and derivatives thereof. Examples of phosphatidylcholines suitable for use in the present invention include dipalmitoylphosphatidylcholine (DPPC), distearoylphosphatidylcholine (DSPC), dimyristoylphosphatidylcholine (DMPC), phosphatidylcholine (PLPC), egg phosphatidylcholine (EPC), dilauryloylphosphatidylcholine (DIPC), dirhyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1-stearoyl-2-palmitoyl phosphatidylcholine (SPPC), palmitoyloeoyl phosphatidylcholine (POPC), lysophosphatidylcholine, dilinoleoylphosphatidylchotine distearoylphophatidylethanolamine (DSPE), dimyristoyl phosphatidylethanoiamine (DMPE), dipalmitoyl phosphatidylethanolamine (DPPE), or a combination thereof.

In a most preferred embodiment, the phosphatidylcholine used in the present invention is selected from the group consisting of dipalmitoylphosphatidyicholine (DPPC), distearoylphosphatidylcholine (DSPC), phosphocholine (DOPC), and dimyristoylphosphatidylcholine (DMPC).

The present invention includes an embodiment in which a combination of at least one neutral lipid that is phosphatidylcholine and at least one additional neutral lipid, cationic lipid, and/or anionic lipid is used to form the lipid bilayers in the VDDS of the present invention.

Preferably, neutral lipids are combined with phosphatidylcholine in the VDDS of the present invention, Examples of neutral lipids that may be used for the present invention include: cholesterol, dipalmitoylphosphatidylcholine (DPPC), distearoylphosphatidylcholine (DSPC), phosphocholine (DOPC), dimyristoylphosphatidylcholine (DMPC), phosphatidylcholine (PLPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1-stearoyl-2- palmitoyl phosphatidylcholine (SPPC), 1,2-distearoyl-sn-glycero-3-phosphocholine (DAPC), 1,2-cliarachidoyl-sn-glycero-3- phosphocholine (DBPC), 1,2-dieicosenoyl-sn-glycero-3-phosphocholine (DEPC), palmitoyloeoyi phosphatidylcholine (POPC), lysophosphatidylcholine, dilinoleoylphosphatidylcholine distearoylphophatidylethanolamine (DSPE), dimyristoyl phosphatidylethanolamine (DMPE), dipalmitoyl phosphatidylethanolamine (DPPE), palmitoyloeoyl phosphatidylethanolamine (POPE), lysophosphatidylethandamine or a combination thereof.

The neutral lipids combined with phosphatidylcholine in the VDDS of the present invention may include polyethylene glycol (PEG)-coupled lipids. Examples of polyethylene glycol (PEG)-coupled lipids that may be used in the present invention include carbonyl methoxypolyethylene glycol-clistearoyl phosphatidyl ethariolamine (IVIPEG-750-DSPE, -MPEG-2000-DSPE and MPEG-5000-DSPE), Carbonyl methoxypolyethylene glycol-dipalmitoyl phosphatidyl ethanolamine (MPEG-2000-DPPE and MPEG-5000-DPPE), Carbonyl methoxypolyethylene glycol-dirnyristoyl phosphatidyl ethanolamine (MPEG-2000-DMPE and MPEG-5000-DMPE) and their derivatives.

In a most preferred embodiment, the neutral lipid is selected from the group consisting of cholesterol, dipalmitoylphosphatidylcholine (DPPC), distearoylphosphatidylcholine (DSPC), phosphocholine (DOPC), dimyristoylphosphatidylcholine (DMPC), and mPEG-2000-DIVIPE.

Suitable cationic lipids for the present invention include, for example, N-1-(2,3-dioleyloxy)propyl-N,N,N-trimethylammonium chloride (DOTMA), 1,2-bis(oleoyloxy)-3-(4′-trimethylammonio)propane (DOTAP), 1,2-dioleoyl-3(4-trimethylammonio)butanoyl-sn-glycerol (DOTB), 1,2-dioleayl-3-succinyl-sn-glycerol choline ester (DOSC), cholesteryl (4′-trimethylammonio)butanoate (ChoTB), 1,2-dioleoyl-3-dimethyl-hydroxyethyl ammonium bromide DORI, 1,2-dioleyloxypropyl-3-dimethyl-hydroxyethyl ammonium bromide (DORIE), 1,2-climyristyloxypropyl-3-dimethyl-hydroxyethyl ammonium bromide (DMRIE), O,O′-didodecyl-N-p-(2-trimethylammonioethyloxy)benzoyl-N,N,N-trimethylam monium chloride, Lipospermine, DC-Chol (3a-N-(N′,N″-climethylaminoethane)carbonylcholesterol), lipopoly(L-lysine), N-(a-trimethylammonioacetyl)-didodecyl-D-glutamate chloride (TMAG), or a combination thereof.

Suitable anionic lipids for the present invention include, for example, phosphatidylserine, phosphatidylglycerol, dimyristoyl phosphatidylserine (DMPS), dipalmitoyl phosphatidylserine (DPPS), brain phosphatidylserine (BPS), dilauryloyiphosphatidylglyceral (DLPG), dimyristoylphosphatidylglycerol (DMPG), dipalrnitoylphosphatidylglycerol (DPPG), distearoyiphosphatidylglycerol (DSPG), dioleoylphosphatidyiglycerol (DOPG), or a combination thereof.

Pharmaceutically acceptable excipients may additionally be used in the VDDS of the present invention. Examples of pharmaceutically acceptable excipients that may be used in the present invention include lipid oxidation inhibitors (U.S. Pat. No. 5,605,703 and WO 9202208), stabilizing agents such as sterols, polyethyleneglycol (PEG) or tocopherol, antioxidants, such as α-tocopherol or its acetate salt; vitamin E: β-carotene; carotenoids, such as α-carotene, lycopene, lutein; zeaxanthine, and the like; and buffering agents such as citrate buffer, tris-buffer, phosphate buffer and the like; or acidifying agents, such as citric acid, maleic acid, oxalic acid, succinic acid, tartaric acid, hydrochloric acid, hydrobrornic acid, phosphoric acid and the like.

Any hydrophobic, i.e., water-insoluble, drug or combination of drugs including at least one hydrophobic drug can be used in the present invention. Suitable drugs include anti-hypertension drugs, antibiotic drugs, and anti-cancer or anti-tumor drugs. Examples of the hydrophobic drugs that may be incorporated into the phospholipid bilayer(s) of the VDD of the present invention include, but are not limited to nitrogenated mustard analogues like cyclophosphamide; melphalan; iphosphamide; or trophospharnide ethyienimines such as thiotepa; nitrosoureas like carmustine: leased agents such as temozolomide; or dacarbazine: analogous antimetabolites of folic acid such as methotrexate or raltitrexed; analogues of purines such as thioguanine, cladribine or fludarabine; analogues of pyrimidines like fluorouracil, tegafur or gemcitabine alkaloids of vinca and analogues such as vinblastine, vincristine or vinorelbine: derivatives of podophyllotoxin such as an etoposide, taxanes, such as docetaxel or paclitaxel; anthracyclines and such as doxorubicin, epirubicin, idarubicin and mitoxantrone; other cytotoxic antibiotics such as Neomycin and mitomycin; platinum compounds such as cisplatin, carboplatin and oxaliplatin; monoclonal antibodies such as rituximato; other antineoplastic agents such as pentostatin, miltefosine, estramustine, topotecan, irinotecan, bicalutamide procarbazine, mechlorethamine, cyclophospharnide, camptothecin, ifosfamide, melphalan, chlorambucil, busulfan, nitrosurea, dactinomycin, daunorubicin, doxorubicin, bleomycin, plicomycin, mitomycin, etoposide (VP 16), tamoxifen, raioxifene, estrogen receptor binding agents, taxol, gemcitabien, navelbine, farnesyl-protein tansferase inhibitors, transplatinum, 5 fluorouracil, viricristin, vinblastin, methotrexate, taxoids, camptothecins, doxorubicin, michellamine B and vincristine, or combinations thereof. The term “taxoid” refers to paclitaxel, cephalomannine, baccatin III, 10-deacetyl baccatin III, deacetylpaclitaxel and deacetyl-7-epipaclitaxel and derivatives and precursors thereof.

In one embodiment of the invention, the hydrophobic drug substance is EPO906 (epothilone B), which is poorly soluble in water (<0.1 mg/l) but highly soluble in organic solvents (ethanol, ethyiacetate, dichloromethane etc). Therefore it is advantageous to incorporate EPO9906 in a VDDS, preferably into a liposomal VDDS to enhance the effective concentration during administration.

Mechanistically, siRNAs and EPO906 differ in their mode of action. EPO906 disrupts the tubulin structures of cells leading to cell death, whereas siRNA can be targeted to specific gene sequences of interest by using the complementary nucleic acid sequence of the mRNA of the gene of interest (“target gene”). Direction of such an siRNA leads to the catalytic degradation of the target mRNA via a multi-protein complex dubbed RISC (RNA induced silencing complex) and therefore, the down-regulation of the protein encoded by this mRNA.

The present invention overcomes the solubility problem of EPO906 and other poorly water soluble drugs by incorporating it into the macromolecular structure of a liposome or multilamellar vesicle. The present invention also overcomes the drawback of the siRNA being rapidly degraded and excreted by incorporating the siRNA into the aqueous interior cage of the liposome or multilamellar vesicle, while simultaneously increasing the pharmacokinetics and pharmacodynamics profile of both drug substances by mediating cellular uptake. This so called “combo” approach of integrating two physicochernically different pharmaceutically active drug substances into one drug delivery overcomes a series of drawbacks of the individual two drug substance such as (i) enhancing the effective local concentration of the poorly water soluble drug, (ii) increasing the protection of both the hydrophobic drug and the siRNA from degradation by hydrolysis and/or specific enzymes (esterases, nucleases), (iii) enhancing the pharmacokinetic and pharmacodynamic profiles of both the drug substances, and (iv) creating a synergistic effect by attacking the target cell from two distinct molecular pathways (e.g., tubulin inhibition in the case of EPO906 and target specific degradation of the mRNA).

The use of siRNAs is well known in the art. In designing a VDDS of the present invention, at least one siRNA is targeted to a specific gene sequence of interest leading to a down-regulation of the protein encoded by this mRNA. Any drugable or non drugable gene of interest may be targeted by designing an siRNA sequence homologous to the mRNA of interest. With the entire human genome now sequenced, any portion thereof may serve as a target sequence in designing an siRNA for use in a VDDS of the present invention. For instance, siRNA could be designed to target an oncogene such as BCI-2, resulting in downregulation and prevention of cancer cell proliferation. Sequences of many oncogenes are known and readily available. Examples include EphA2, focal adhesion kinase (FAK), β₂-adrenergic receptor (β₂AR), ESR1 , tumor suppressor protein pRB, MDR-1, NKkappaB and Nek2.

Thus, the sequence for an siRNA for use in the present invention may be obtained from plasma DNA or the human genorne. siRNA may be generated by ribonuclease Ill cleavage from longer double-stranded RNA (dsRNA) which are homologous to the silenced gene or by delivering synthetic RNAs to cells. The target gene sequence may be drugable or non-drugable. siRNAs for use in the VDDS of the present invention may also be derived from known anti-sense sequences.

The siRNA for use in the present invention are double stranded ribonucleotide sequences of typically 15 to 50 base pairs in length that are highly negatively charged and soluble predominantly in water. Preferably, the siRNA sequence of the present invention ranges from about 19 to about 27 base pairs in length and is highly homologous or 100% homologous to the target sequence. The siRNA may be blunt ended or else have base pair overhangs. The siRNA further may include a repeating amino acid sequence consisting of serine-aspartic acid-threonine and/or phosphorothioate backbone modifications,

Various types of RNA may serve as siRNA for use in a VDDS of the present invention. For example, double stranded RNA (dsRNA), micro-RNA (miRNA), short hairpin RNA (shRNA) or combinations thereof, may be used.

The ratio of lipid to siRNA used in the present invention may range from 10:1 (mass) to 20:1 (mass). Preferably, the ratio of lipid to siRNA, used in the present invention is approximately 14:1.

In one embodiment of the invention, the VDDS and pharmaceutical compositions including the VDDS of the present invention can be formulated into a variety of suitable formulations and administered orally, in aerosol form, parenterally, subcutaneously, intravenously, intramuscularly, intraperitoneally, interperitoneally, rectally, topically and vaginally.

Preferably, the VDDS is admixed with a pharmaceutically acceptable carrier and administered intravenously (iv), intraperitoneally (ip) or topically (tp). Administration by iv or ip results in a reduction in the risk of side effects such as inflammation of the injection site.

In accordance with the present invention, a “targeting moiety” or ligand may be attached to the VDDS for recognizing target tissue and thus, improving intracellular delivery of drug substances delivered by VDDSs, for example, antibodies, antibody fragments, polysaccharides, sugars, and other ligands or other agents and/or methods known to those of ordinary skill in the art (Klibanov et al., J. Liposome Res., 2(3):321 (1992)). Gangliosides, polysaccharides and polymers such as polyethylene glycol can be attached to VDDSs to decrease their non-specific uptake by the reticuloendothelial system in vivo. Various cellular and viral proteins have also been incorporated into liposomes for targeting purposes and for thew fusogenic properties, According to one embodiment of the present invention, the targeting moiety is coupled to a hydrophilic polymer (e.g., PEG), in order to modify the targeting specificity. According to one embodiment of the present invention, the targeting moiety is folate-mPEG-DSPE transferrin, alpha-tocopherol, retinoic acid or RGD peptide.

The present invention provides a method of making a VDDS comprising (a) at least one lipid bilayer comprising a neutral lipid phosphatidylcholine enciosing at least one aqueous cavity; (b) at least one short interfering ribonucleic acid (siRNA) contained within said at least one aqueous cavity; and (c) at least one hydrophobic drug substance embedded in said at least one lipid bilayer. The method comprises the steps of (i) forming a first solution comprising a neutral lipid phosphatidyicholine, at least one hydrophobic drug and a water-miscible organic solvent, (ii) forming a lipid film by evaporating the water-miscible organic solvent from the first solution, (iii) forming a second solution comprising at least one short interfering ribonucleic acid (siRNA) and an aqueous solution, and (iv) combining the lipid film of (ii) with the second solution of (iii) to form the VDDS.

The ratio of lipid to siRNA used to make the VDDS of the present invention may range from 10:1 (mass) to 20:1 (mass). Preferably, the ratio of lipid to siRNA used in the present invention is approximately 14:1.

The “water-miscible organic solvent” used in the present invention is selected depending on the solubility of the hydrophobic drug in the solvent, the degree to which the solvent is miscible in water and the toxicity of the solvent. Examples of solvents that may be used include, but are not limited to, dimethylsulfoxide (DMSO), dimethylacetarnide (DMA), dimethylformamide, various alcohols such as ethanol, glycols, glycerin, propylene glycol, and various polyethylene glycols. According to one embodiment of the present invention, the method of making the VDDS uses ethanol as the water-miscible organic solvent to form a mixture comprising the at least one phospholipid and at least one hydrophobic drug to be further mixed with the aqueous solution comprising at least one short interfering ribonucleic acid (siRNA) to form the VDDSS.

The aqueous solution used to form the second solution containing siRNA includes both a buffer at a pH between 3-6, preferably ph of 4, and a metal salt. This aqueous solution is selected depending on the solubility of the siRNA. One of ordinary skill would understand how to select a buffer and metal salt for the aqueous solution in the present invention. Examples of suitable buffers include any salt of acetic acid, including sodium acetate and potassium acetate, succinate buffer, phosphate buffer, citrate buffer, HEPES, PBS and any others known to the art. Examples of suitable metal salts include calcium chloride, zinc chloride and magnesium chloride.

Additives to the VDDS that enhance stability or reduce the toxicity of the drug substances can be added such as lipid oxidation inhibitors (U.S. Pat. No. 5,605,703 and WO 9202208) and stabilizing agents such as sterols, polyethyleneglycol (PEG) or tocopherol. Sterols, such as cholesterol, when included in a VDDS, promote stability by making the bilayer(s) less permeable to small molecules and ions and reducing the traffic of proteins between the bilayers.

A “targeting moiety” or ligand for recognizing target tissue may be attached to the VDDS using methods known to those of ordinary skill in the art (Klibanov et at J. Liposome Res., 2(3):321 (1992)). These known methods are hereby incorporated by reference.

Removal of free, non-liposomal drug and siRNA may be achieved using a tangential (membrane) flow filtration (TFF) system or dialysis.

The present invention further provides a method of making a VDDS comprising (a) at least one lipid bilayer comprising a combination of a neutral lipid phosphatidylcholine and at least one additional neutral lipid, cationic lipid and/or anionic lipid enclosing at least one aqueous cavity; (b) at least one short interfering ribonucleic acid (siRNA) contained within said at least one aqueous cavity; and (c) at least one hydrophobic drug substance embedded in said at least one lipid bilayer. The method comprises the steps of: (i) forming a first solution comprising the neutral lipid phosphatidylcholine, the additional neutral or cationic or anionic lipid(s), at least one hydrophobic drug and a water-miscible organic solvent, (ii) forming a lipid film by evaporating the water-miscible organic solvent from the first solution, (iii) forming a second solution comprising at least one short interfering ribonucleic acid (siRNA) and an aqueous solution, and (iv) combining the lipid film of (ii) with the second solution of (iii) to form the VDDS.

The ratio of lipid to siRNA used to make the VDDS of the present invention may range from 10:1 (mass) to 20:1 (mass). Preferably, the ratio of lipid to siRNA used in the present invention is approximately 14:1.

A preferred formulation of a VDDS of the present invention has a ratio of PC:chol:DMPE-PEG2000:D,L-α-tocopherol (Vitamin E) of 81.9:15.4:2:0.7 (molar), wherein “PC” is phosphatidylcholine, “chol” is cholesterol and “DMPE-PEG2000” is dirnyristylphosphatidylethanolamine-polyethyleneglycol 2000. A preferred ratio of lipid-to-siRNA is 14:1 (mass). The amount of drug intercalating into the liposomal bilayer depends on the particular drug. In the case of EPO906, a final concentration of 0.5 mg/niL intercalating into the bilayer is preferred, siRNA accumulation in the aqueous interior of the liposome is preferably at a final concentration of about 1-1.2 mg/mL.

The present invention further provides a method of simultaneous co-delivery of at least one hydrophobic drug substance and at least one siRNA to a subject, said method comprising administering to the subject a vesicular drug delivery system which comprises: (a) at least one lipid bilayer enclosing at least one aqueous cavity; (b) at least one short interfering ribonucleic acid (siRNA) contained within said at least one aqueous cavity; and (c) at least one hydrophobic drug substance embedded in said at least one lipid bilayer. In accordance with the present invention, the lipid Weyer comprises a neutral lipid that is phosphatidylcholine. The present invention further includes an embodiment in which the lipid bilayer comprises a combination of phosphatidylcholine and at least one additional neutral lipid, cationic lipid and/or anionic lipid.

A VDDS of the present invention is useful in the treatment of various diseases, especially in oncology applications. For example, a VDDS comprising EPO906 and a siRNA may be used for treating ovarian cancer, where administration is preferably intraperitoneal or systemic/parenteral administration. A VDDS of the present invention comprising EPO906 and a siRNA may also be used in the treatment of breast, lung, melanoma, peritoneal, fallopian, and colorectal cancer. A preferred dose range is from 0.1 to 10 mg/kg siRNA per individual application which equates to 0.05 to 5 mg/kg EPO906 per individual application,

The following example is illustrative but does not serve to limit the scope of the invention described herein. The example is meant only to suggest a method of practicing the invention.

### EXAMPLE 1

Preparation of lipid film without a targeting moiety:

Phosphatidylcholine (PLPC, 104A mg), cholesterol (10 mg), MPEG-2000-DMPE (9 mg), and D,L-α-tocopherol (0.49 mg) are dissolved in 2 mL ethanol to give 62.01 mg/mL (or 0.0840 mmol/mL), EPO906 (22.14 mg) is added and completely dissolved From this solution, 1129 μL is pipetted into a separate round-bottomed flask and all ethanol is evaporated to generate a lipid film.

Preparation of lipid film with a targeting moiety:

The targeting moiety used in this example is FA-mPEG-DSPE, Phosphatidylcholine (PLPC, 104.1 mg), cholesterol (10.22 mg), mPEG-2000-DMPE (9 mg), FA-PEG3000-DSPE (2.2 mg) and D,L-α-tocopherol (0.52 mg) are dissolved in 2mL ethanol to give 62.92 mg/mL (or 0,084 mmol/mL). EPO906 (22.46 mg) is added and completely dissolved. From this solution, 1113 μL is pipetted into a separate round-bottomed flask and ethanol is evaporated to generate a lipid film.

Preparation of siRNA aqueous solution:

In this example, a luciferase gene specific siRNA is used. A siRNA solution of 1000 μL siRNA stock (5 mg/mL), 125 μL CaCl₂ (0.1 M), 125 μL acetic acid buffer solution (pH 4, 1 M) and 250 μL water is prepared by mixing. The siRNA solution is warmed in a 45° C. water bath,

### Preparation of VDDS:

The particular lipid film is mixed and hydrated with the siRNA aqueous solution to form a lipoplex solution. The lipoplex solution is left in a 45° C. hot water bath for 30 min before addition of 1000 μL ethanol. The lipoplex solution is frozen using liquid nitrogen and warmed in the 45° C. hot water bath. The step of freezing and warming is repeated three times in order to increase the trapping efficiencies for the water soluble drug substances resulting in a VDDS of the present invention. Analysis of the particles shows a mean size of 140±56 nm and a PDI of 0.5 with encapsulation efficiencies of 80% as determined by gel electrophoresis,

Removal of free, non-liposomal EPO906 and siRNA is achieved using a tangential (membrane) flow filtration (TFF) system.

1. A vesicular drug delivery system comprising:
a) at least one lipid bilayer enclosing at least one aqueous cavity, wherein the lipid comprises at least one neutral lipid that is phosphatidylcholine;
b) at least one short interfering ribonucleic acid (siRNA) contained within said at least one aqueous cavity; and
c) at least one hydrophobic drug substance embedded in said at least one lipid bilayer.

2. The vesicular drug delivery system of claim 1, wherein the lipid further comprises at least one additional neutral lipid, cationic lipid or anionic lipid.

3. The vesicular drug delivery system of claim 1 wherein the at least one siRNA comprises double-stranded RNA (dsRNA), micro-RNA (miRNA), short hairpin RNA (shRNA), or combinations thereof.

4. The vesicular drug delivery system of claim 1 wherein the nucleotide sequence of said siRNA is derived from an antisense oligonucleotide, plasma DNA or human genome.

5. The vesicular drug delivery system of claim 1 wherein the hydrophobic drug is selected from the group consisting of a taxoid, docetaxel, paclitaxel, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP 16), tamoxifen, raloxifene, estrogen receptor binding agents, taxol, gemcitabien, navelbine, farnesyl-protein tansferase inhibitors, transplatinum, 5-fluorouracil, vincristin, vinblastin, methotrexate, cyclosporine, michellamine B, bryostatin-1, halomon, cisplatin, EPO906 or a combination thereof.

6. The vesicular drug delivery system of claim 5 wherein the hydrophobic drug is EPO906.

7. The vesicular drug delivery system of claim 2, wherein said at least one additional neutral lipid is cholesterol, dipalmitoylphosphatidylcholine (DPPC), distearoylphosphatidylcholine (DSPC), phosphocholine (DOPC), dimyristoylphosphatidylcholine (DMPC), phosphatidylcholine (PLPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1-stearoyl-2-palmitoyl phosphatidylcholine (SPPC), 1,2-distearoyl-sn-glycero-3-phosphocholine (DAPC), 1,2-diarachidoyl-sn-glycero-3-phosphocholine (DBPC), 1,2-dieicosenoyl-sn-glycero-3-phosphocholine (DEPC), palmitoyloeoyl phosphatidylcholine (POPC), lysophosphatidylcholine, dilinoleoylphosphatidylcholine distearoylphophatidylethanolamine (DSPE), dimyristoyl phosphatidylethanolamine (DMPE), dipalmitoyl phosphatidylethanolamine (DPPE), palmitoyloeoyl phosphatidylethanolamine (POPE), lysophosphatidylethanolamine, a carbonyl methoxypolyethylene glycol-distearoyl phosphatidyl ethanolamine (MPEG-750-DSPE, -MPEG-2000-DSPE and MPEG-5000-DSPE), carbonyl methoxypolyethylene glycol-dipalmitoyl phosphatidyl ethanolamine (MPEG-2000-DPPE and MPEG-5000-DPPE), carbonyl methoxypolyethylene glycol-dimyristoyl phosphatidyl ethanolamine (MPEG-2000-DMPE and MPEG-5000-DMPE) or a combination thereof.

8. The vesicular drug delivery system of claim 7, wherein the neutral lipid is DPPC, DSPC, DOPC, DMPC, PLPC, PE, MPEG-2000-DMPE or a combination thereof.

9. The vesicular drug delivery system of claim 2, wherein said at least one additional cationic lipid is N-1-(2,3-dioleyloxy)propyl-N,N,N-trimethylammonium chloride (DOTMA), 1,2-bis(oleoyloxy)-3-(4′-trimethylammonio)propane (DOTAP), 1,2-dioleoyl-3-(4′-trimethylammonio)butanoyl-sn-glycerol (DOTB), 1,2-dioleoyl-3-succinyl-sn-glycerol choline ester (DOSC), cholesteryl (4′-trimethylammonio)butanoate (ChoTB), 1,2-dioleoyl-3-dimethyl-hydroxyethyl ammonium bromide DORI, 1,2-dioleyloxypropyl-3-dimethyl-hydroxyethyl ammonium bromide (DORIE), 1,2-dimyristyloxypropyl-3-dimethyl-hydroxyethyl ammonium bromide (DMRIE), O,O′-didodecyl-N-p-(2-trimethylammonioethyloxy)benzoyl-N,N,N-trimethylam monium chloride, Lipospermine, DC-Chol (3a-N-(N′,N″-dimethylaminoethane)carbonylcholesterol), lipopoly(L-lysine), N-(a-trimethylammonioacetyl)-didodecyl-D-glutamate chloride (TMAG), or a combination thereof.

10. The vesicular drug delivery system of claim 2, wherein said at least one additional anionic lipid is phosphatidylserine, phosphatidylglycerol, dimyristoyl phosphatidylserine (DMPS), dipalmitoyl phosphatidylserine (DPPS), brain phosphatidylserine (BPS), dilauryloylphosphatidylglycerol (DLPG), dimyristoylphosphatidylglycerol (DMPG), dipalmitoylphosphatidylglycerol (DPPG), distearoylphosphatidylglycerol (DSPG), dioleoylphosphatidylglycerol (DOPG), or a combination thereof.

11. The vesicular drug delivery system of claim 1 further comprising an additional pharmaceutically acceptable excipient.

12. The vesicular drug delivery system of claim 11, wherein additional pharmaceutically acceptable excipient is a stabilizing agent selected from the group consisting of cholesterol, polyethyleneglycol (PEG), or tocopherol.

13. The vesicular drug delivery system of claim 1 wherein the siRNA inhibits the translation of a gene that promotes growth of a hyperplastic or cancerous cell.

14. The vesicular drug delivery system of claim 1 wherein the siRNA is 15 to 50 nucleobases.

15. The vesicular drug delivery system of claim 13 wherein the gene is selected from the group consisting of EphA2, focal adhesion kinase (FAK), [beta]2 adrenergic receptor ([beta]2AR), ESR1, tumor suppressor protein pRB, MDR-1, NKkappaB and Nek2.

16. The vesicular drug delivery system of claim 1 further comprising a targeting moiety.

17. The vesicular drug delivery system of claim 16 wherein the targeting moiety is selected from the group consisting of folate-mPEG-DSPE transferrin, alpha-tocopherol, retinoic acid and RGD peptide.

18. The vesicular drug delivery system of claim 9 further comprising cholesterol, dimyristoyl phosphatidylethanolamine polyethylene glycol 2000 (DMPE-PEG2000) and tocopherol.

19. The vesicular drug delivery system as in claim 18 wherein the ratio of PC:cholesterol:DMPE-PEG2000:tocopherol is about 81.9:15.4:2:0.7 (molar).

20. The vesicular drug delivery system as in claim 1 wherein the ratio of lipid:siRNA is ranging between 10:1 (mass) and 20:1 (mass).

21. The vesicular drug delivery system as in claim 20 wherein the ratio of phospholipid:siRNA is 14:1 (mass).

22. The vesicular drug delivery system as in claim 1 wherein the concentration of hydrophobic drug is at least about 0.5 mg/mL.

23. The vesicular drug delivery system as in claim 1 wherein the concentration of siRNA is about 1-1.2 mg/mL.

24. A method of making the vesicular drug delivery system of claim 1, said method comprising:
a) forming a first solution comprising at least one neutral lipid that is phosphatidylcholine, at least one hydrophobic drug and a water-miscible organic solvent,
b) forming a lipid film by evaporating the water-miscible organic solvent from the first solution,
c) forming a second solution comprising at least one short interfering ribonucleic acid (siRNA) and an aqueous solution,
d) combining the lipid film of (b) with the second solution of (c) to form the vesicular drug delivery system.

25. A method of making the vesicular drug delivery system of claim 2, said method comprising:
a) forming a first solution comprising at least one neutral lipid that is phosphatidylcholine; at least one additional neutral lipid, cationic lipid or anionic lipid; at least one hydrophobic drug and a water-miscible organic solvent;
b) forming a lipid film by evaporating the water-miscible organic solvent from the first solution;
c) forming a second solution comprising at least one short interfering ribonucleic acid (siRNA) and an aqueous solution; and
d) combining the lipid film of (b) with the second solution of (c) to form the vesicular drug delivery system.

26. The method of claim 24, wherein a targeting moiety is added to the first solution of (a).

27. The method of claim 24, wherein said hydrophobic drug is a taxoid, camptothecin, doxorubicin, michellamine B, vincristine, bryostatin-1, halomon, cisplatin, EPO906, docetaxel, paclitaxel, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP 16), tamoxifen, raloxifene, estrogen receptor binding agents, taxol, gemcitabien, navelbine, farnesyl-protein tansferase inhibitors, transplatinum, 5- fluorouracil, vincristin, vinblastin, methotrexate, and cyclosporin, or a combination thereof.

28. The method of claim 24, wherein said water-miscible organic solvent is selected from the group comprising of an alcohol, acetone, dimethylformamide, dimethylsulfoxide, dimethylacetamide or a polyethylene glycol.

29. The method of claim 28, wherein the water-miscible organic solvent is an alcohol selected from the group comprising of methanol, glycol, glycerol, propylene glycol or ethanol.

30. The method of claim 24, wherein said at least one additional neutral lipid is selected from the group comprising of dipalmitoylphosphatidylcholine (DPPC), distearoylphosphatidylcholine (DSPC), phosphocholine (DOPC), dimyristoylphosphatidylcholine (DMPC), phosphatidylcholine (PLPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1-stearoyl-2-palmitoyl phosphatidylcholine (SPPC), 1,2-distearoyl-sn-glycero-3-phosphocholine (DAPC), 1,2-diarachidoyl-sn-glycero-3- phosphocholine (DBPC), 1,2-dieicosenoyl-sn-glycero-3-phosphocholine (DEPC), palmitoyloeoyl phosphatidylcholine (POPC), lysophosphatidylcholine, dilinoleoylphosphatidylcholine distearoylphophatidylethanolamine (DSPE), dimyristoyl phosphatidylethanolamine (DMPE), dipalmitoyl phosphatidylethanolamine (DPPE), palmitoyloeoyl phosphatidylethanolamine (POPE), and lysophosphatidylethanolamine or a combination thereof.

31. The method of claim 24, wherein a stabilizing agent is added to the first solution of (a).

32. The method of claim 31 wherein the stabilizing agent is cholesterol, polyethyleneglycol (PEG) or tocopherol.

33. A method of simultaneous co-delivery of a hydrophobic drug substance and a siRNA to a subject, said method comprising administering to the subject a vesicular drug delivery system of claim 1.
